(19) United States
(12) Patent Application Publication
(10) Pub. No.: US 2016/0067299 A1
(43) Pub. Date: Mar. 10, 2016

(54) USE OF DIETARY FIBRE SUPPLEMENT IN A FOOD FORMULATION

(71) Applicant: GRATUK TECHNOLOGIES PTY LTD, Sydney, New South Wales (AU)

(72) Inventors: Malcolm BALL, Sydney (AU); Gordon EDWARDS, Sydney (AU)

(21) Appl. No.: 14/782,456

(22) PCT Filed: Apr. 7, 2014
(86) PCT No.: PCT/IB2014/060471

§ 371 Date: Oct. 5, 2015

(30) Foreign Application Priority Data
Apr. 5, 2013 (AU) 2013901175

(51) Int. Cl. A61K 36/899; A23L 1/308; A61K 9/00

(52) U.S. Cl. CPC A61K 36/899; A61K 9/0095; A61K 9/0056; A23L 1/3082; A23V 2002/00

(57) ABSTRACT

Use of dietary fibre material extracted from sugarcane in the manufacture of a food product that is formulated to ameliorate the effects of intestinal tract disorders such as Coeliac's disease and Irritable Bowel Syndrome (IBS).

### TECHNICAL FIELD

The invention relates to the field of food and food supplement manufacture. In particular, the invention relates to a dietary fibre supplement, the use of food products containing said supplement and its method of manufacture.

### BACKGROUND OF THE INVENTION

Consumption of dietary fibre is an essential part of healthy digestive function. It aids in the absorption of nutrients, in feeding gut flora, and in physically scouring the intestine to help turn over the lining of the lumen.

Too little consumption of dietary fibre has been linked to a number of conditions such as constipation, diarrhoea, and even colorectal cancer. The World Health Organisation (WHO) has estimated that the average person in the developed world is deficient in their daily dietary fibre intake by as much as 15 g.

This problem is of particular concern for the subset of the population that suffer from digestive disorders, such as Coeliac disease and irritable bowel syndrome (IBS). An individual with Coeliac disease has a negative reaction to the gluten protein found in some grains such as wheat, rye, barley, and oats. It has been calculated that 1.0-1.5% of the Caucasian population have Coeliac disease, with the numbers for other ethnic groups still under investigation. Gluten intolerance has many degrees of severity, however, and it is estimated that up to 10% of the human population may be gluten intolerant to some degree. The most common symptoms of this in adults can include:
Anaemia;
Bloating and flatulence;
Diarrhoea or constipation;
Fatigue, weakness and lethargy;
Nausea and vomiting;
Stomach cramps; and
Unwanted weight loss or weight gain.

The most common symptoms in children include:
Abdominal pain, bloating and flatulence;
Bulky, foul-smelling bowel motions;
Chronic anaemia;
Diarrhoea or constipation;
Nausea and vomiting;
Poor weight gain or weight loss in older children;
Delayed growth or delayed puberty;
Tiredness; and
Irritability.

One of the major concerns for Coeliac disease sufferers (‘Coeliacs’) is that the majority of grains that contain dietary fibre also contain gluten. This means that gluten-free flour based products usually contain little or no fibre. Coeliacs therefore suffer two-fold in that their digestive tract already functions sub-optimally, and they are also lacking sufficient dietary fibre to maintain normal gut function. Though fibre can be found in many woody vegetables it is still extremely difficult for someone who is intolerant to gluten to consume enough dietary fibre.

Sufferers of IBS also have problems with many sources of fibre though for different reasons. A symptom associated with IBS is that the lining of the gut becomes inflamed making it uncomfortable or painful to digest food. IBS often results in constipation which is difficult to treat on a long term basis due to sensitivity to many forms of fibre.

One source of fibre commonly used for these two risk groups is psyllium husk. While this fibre does not contain gluten and is relatively gentle to the intestines it does have several drawbacks:
1. It is composed almost entirely from soluble fibre, which does not mimic the natural constituents of dietary fibre from a normal diet.
2. It is difficult to use, e.g. it cannot readily be formulated into a pre-made drink as it forms a ‘gel-like’ consistency when mixed with liquids.
3. Prolonged consumption can lead to blockages of the oesophagus, stomach and/or gut.
4. Many people who suffer from grass allergies are also allergic to this source of fibre. Several cases of adverse events and even anaphylaxis have been reported in users.

Accordingly, it is an object of the invention to improve the delivery of dietary fibre to the diet of persons suffering from intestinal disorders, such as Coeliac disease, IBS, etc., that ameliorates the identified disadvantages of the prior art.

### SUMMARY OF THE INVENTION

According to one aspect of the invention, there is provided the use of dietary fibre material extracted from sugar cane in the manufacture of a food product that is formulated to ameliorate the effects of intestinal tract disorders such as Coeliac's disease and Irritable Bowel Syndrome (IBS).

Preferably, the sugar cane fibre is prepared via a process including the steps of: subjecting the sugar cane material to at least one wet diffusion step to separate sugars from a residual fibre material; and subjecting the residual fibre material to a rapid, low-heat drying process thereby to retain the biologically active molecules in the fibre, and to enhance the water retention properties of said residual fibre product.

There are a number of advantages to using this particular fibre source in the way described above. Firstly, no adverse allergic effects have ever been recorded with this source. Also, this fibre source has been shown to improve gut lining health over and above other sources of fibre. It contains benefits of both soluble and insoluble fibre and has a ratio of fibres that more accurately represents natural foods than other products. It is also high in other micronutrients such as iron and has the ability to protect antioxidants.

In addition, when this fibre source is prepared via the process as described herein, the fibre tends to retain the active ingredients to a greater level. The fibre source also provides the correct dietary fibre to address the deficiency in the majority of the population.

The invention also allows more flexible product formats to be developed, in particular that allow sufferers of gut health problems to address to deficiency in their own way, especially when provided with the correct type of fibre in a relatively easy-to-use format. Sufferers no longer have to rely on food manufacturers to generate high fibre foods that they can eat.

Preferably, the wet extraction step is a counter-current extraction, done under relatively low-shear conditions. The optimal wet extraction step temperature is in the range 25° C. to 70° C.

Other preferable process conditions include wherein the sugar cane fibre material has undergone a pressure heating step, at a pressure in the range 100 psi to 140 psi, and a temperature the range 100° C. to 250° C.

According to another aspect of the invention, there is provided a food product formulated to ameliorate the effects of intestinal tract disorders such as Coeliac's disease and IBS; said food product containing dietary fibre material extracted from sugar cane, said dietary fibre material preferably having been prepared according to the steps defined above.

Food products according to the invention are particularly aimed at those people that are unable to use traditional sources of fibre, such as wheat bran, due to pre-existing conditions such as coeliac disease or IBS, but are equally applicable to all individuals that are not consuming their daily allowance of fibre.

According to another aspect of the invention, there is provided a method of treatment of the effects of intestinal tract disorders such as Coeliac's disease and IBS in a person by feeding to said person a food product incorporating dietary fibre material extracted from sugar cane said dietary fibre material preferably having been prepared according to the steps defined above.

Now will be described, by way of particular, non-limiting examples, preferred embodiments of the invention.

### DETAILED DESCRIPTION OF THE INVENTION

The current invention takes advantage of the properties of a dietary fibre isolate produced from sugar cane, in such a way that maximised retention and minimal destruction of the bioactive molecules occurs.

The method of preparation of the fibre material from sugar cane is broadly similar to that described in WIPO patent document no. WO2011/035381 by KFSU Pty Ltd, which is incorporated herein by reference. However, the process according to the present invention may be defined as having the following essential features:
1. A sugar cane size reduction step;
2. A relatively ‘gentle’ aqueous extraction stage that separates the fibre from other sugar cane fractions, including the sugar fraction, without causing degradation of the fibre functionality; and
3. A relatively gentle drying step that minimises degradation of the fibre functionality.

It is preferred that the extraction step be a counter-current aqueous extraction performed at a relatively neutral pH. It is also preferred that the drying step be a rapid vortex drying operation that, as may be achieved via a low temperature, vortex dryer, such as that supplied by Tensei in Japan (www.tensei-j.com).

It is understood that dietary fibre is important to the correct function of the digestive system. It is also thought that most Western diets are deficient in dietary fibre. This is particularly so for sufferers of disorders such as Coeliac disease and Irritable Bowel Syndrome (IBS).

Sugarcane fibre has a number of advantages over other dietary fibre sources in that:
It is relatively hypoallergenic;
It contains both insoluble and soluble fibre in beneficial proportions for dietary intake;
It contains a number of bioactive molecules that affect blood glucose levels and intestinal health to a greater degree than other fibre sources;
It can be prepared in a ‘chemical-free’ manner and contains no harmful trace elements, unlike fibre from sources such as chemically modified starch; and
It can be prepared in such a way as to retain the micronutrients and active molecules found in the “molasses” component of sugarcane;

While this fibre source is particularly suited to gluten-intolerant individuals and those with intestinal disorders, the diet of most of the population of the developed world is deficient in dietary fibre, and so the invention is widely beneficial.

In another embodiment of the invention, the extracted sugarcane fibre is provided as a concentrated supplement that can either be taken with a meal or added to any food. This allows the level of the individual's dietary fibre intake to be more easily controlled by the individual. The supplement is also classed as a natural food, a status which is increasingly important to many consumers.

The embodiments of the invention can take a number of forms, each with several advantages for users.

In this document:
The term “carrier” is used to describe a palatable ingredient that can be combined with the sugarcane fibre to make consumption of the fibre easier for the individual. The carrier may contain protein or other nutrients, including, but not limited to, juices, puddings, sauces, and yogurts.
The term “inert filler” refers to any product used to add bulk to the sugarcane fibre to allow for ease of packaging or consumption. The inert filler may contain flavours or nutrient, but would not significantly contribute to the total fibre content of the invention.
The term “pellet” refers to any pressed form of the fibre, including, but not limited to: a dried pill; a “grain”-style sprinkle; a compacted powder that may be added to the medium of the user's choice.

All of the examples below can optionally be formulated with additional vitamins, sweeteners, and bioactive molecules such as stevia. Preferably any added nutrients would be sourced from natural ingredient to that the “natural” descriptor may be maintained for the final product.

### EXAMPLE 1

The sugarcane fibre may be added to a flavoured aqueous medium such as juice, soy milk, or milk. The fibre is dispersed into the media at a ratio of 0.1-5.0 g per hundred ml, based on the desired dose. If the consumer is particularly sensitive to fibre due to a pre-existing gut condition then dosage may be administered in stages.

The first stage of consumption would be 1 g of fibre per serve (80-90 ml); second stage would be 2 g of fibre per serve; the third stage 3 g per serve and finally 4-5 g per serve. The drink is then packaged into single serve, daily (3 serve) or two-daily (6 serve) packages and pasteurised for storage.

The consumer could then take as many serves per day as was required to address their fibre deficiency (3 serves on average would largely address the World Health Organisation's calculated average fibre deficiency). Sweeteners, flavours and other nutrients may be added as required.

### EXAMPLE 2

In this example the fibre product is added to a semi solid food product, such as a pudding or yogurt. As per example 1, the fibre is dispersed into the food at a ratio of 0.1-5.0 g per hundred ml based on the desired dose.

If the consumer is particularly sensitive to fibre due to a pre-existing gut condition then dosage would be administered in stages. The first stage of consumption would be 1 g of fibre product per serve (80-150 ml); second stage would be 2 g of fibre product per serve; third stage 3 g per serve and finally 4-5 g per serve.

The pudding/yogurt is then packaged into single serve containers for ease of consumption. The consumer could then take as many serves per day as is required to address their fibre deficiency (3 serves on average would largely address the World Health Organisation's calculated average fibre deficiency). Sweeteners, flavours and other nutrients may be added as desired.

### EXAMPLE 3

In this example the sugarcane fibre supplement is prepared as powder to be added to other meals, drinks or confectionery. The fibre would be mixed with a dry flavour component and an inert filler to form easy-to-use granules. The dose (1.0-5.0 g) would be equal to one scoop, or one single serve sachet, of the fibre product.

As with examples 1 and 2 above, the serves may be increased in stages for new users or those with particularly sensitive conditions. Alternatively the fibre material could be pressed into an easy-to-swallow tablet or dissolvable cube, and flavoured as desired.

### EXAMPLE 4

In this example the fibre material is supplied as an ingredient for other manufacturers of high-fibre foods for the gluten-free or ‘digestive eating’ markets. This example provides several benefits for potential food manufacturers/suppliers:
If the fibre material is used to replace wheat or oat fibres then the product may be labelled as hypo-allergenic.
The fibre material supports the use of “all natural” marketing claims for the foods.
The fibre material provides other health benefits compared with other fibre sources, allowing the food manufacturer to potentially make more substantive claims.

The water retention capacity of the sugar cane fibre, prepared as described above, is far greater than most commercial fibre sources. By using the active fibre in foods the manufacturer can reduce calorific content per kg of food. There may also be a significant commercial saving for the manufacturer.

In example 4 the fibre material would be supplied to food manufacturers without further modification.

Food products and methods according to the invention make use of the unique qualities of cane based crops, particularly whole sugarcane, that have been prepared using a chemical-free, low-heat procedure. This makes it easy and convenient to use while still retaining the beneficial nutrients and bioactive molecules in the food.

The products and methods address several problems associated with poor fibre consumption as well as having a positive impact on gut health, while also contributing to the elimination of the potential problems of intolerance and malabsorption in individuals who suffer allergies or intolerances to common fibre sources. The product may also be classed as a natural, whole food meaning it does not have some of the problems associated with many pharmaceutical treatments, including negative side effects.

When incorporated in an existing food product format, such as in examples 1-3 above, the fibre product described above potentially provides the following benefits to the overall food product:
a) Tends to increase the fibre content of the food;
b) May acts as a bulking agent, increasing yield;
c) Adds moisture to drier formulations (as is commonly the case with gluten-free recipes);
d) Is allergen-free; and
e) Tends to reduce the G.I. of the food.

In an alternative form, as per example 4 above, the fibre product can be sold as a stand-alone additive that can be used in conjunction with the individuals' normal diet, which allows the following advantages:
a) Can be sold in single or multiple serve packaging for convenience of use;
b) May be prepared as a drink (juice or milk based), pudding, or individual sachet;
c) The dosage can be varied as required for individuals with particularly sensitive digestive systems;
d) May be taken with normal meals to aid digestion and improve general feelings of wellbeing;
e) May be a combined with other natural products to enhance the digestive health properties of the individual's diet; and
f) Tends to reduce the GI of any food it is taken with.

It is envisaged that each serve of food product incorporating the fibre product will contain a single “dose” based on WHO recommendations for healthy fibre intake, with 3 doses a day (one at each major meal) totalling the average fibre deficiency of individuals in the industrialised world.

The supplement tends to provide several benefits to the consumer, including increasing the fibre content of the food, having a positive effect on digestion, prolong control of blood glucose levels, lower blood lipid levels and reduced irritation of the gut. By using the supplement in this form the low-fibre foods that are typical for gluten free diets may be improved for the user.

Additionally, as the fibre source is relatively ‘gentle’ to the digestive system, it may be used as a suitable dietary fibre source for sufferers of IBS and other digestive disorders that would normally require low fibre diets.

### EXAMPLE 5

The acute and long term benefits of the consumption of sugarcane fibre according to the invention on the digestive health of individuals was monitored. The particular aim of the study was to determine if addition of said sugarcane fibre to the diet of a sufferer of digestive dysfunction improves the subject's health outcomes.

In this example; a 35 year old male was presenting with multiple digestive issues, experiencing periods of digestive distress and discomfort, bloating, irregularity, oesophageal irritation and suspected Gastro Oesophageal Reflux Disease (GORD). The individual had been under practitioner care for these issues for approximately 15 years as lifestyle and diet increased and decreased the symptoms.

Treatment options offered and prescribed many times to this patient were NEXIUM™ (esomeprazole) and over-the-counter (OTC) retail antacids. While the pharmaceutical medication helped to ease the symptoms it also resulted in headaches and light-headed' sensations. The individual preferred to live with the intestinal distress rather than the side effects of esomeprazole and therefore discontinued use. While OTC antacids provided some relief the subject was using them at far higher than recommended rates with unsatisfactory benefits.

The individual began trialling the sugarcane fibre according to the invention as a general supplement to dietary intake. They found that after only 1 week of regular instructed usage the symptoms eased to the extent that the individual reported a complete remission of all symptoms for extended periods of time. The subject was also able to tolerate ‘trigger’ foods, such as alcohol or spices, without concern or acute symptoms.

The individual has reported that the dietary fibre material extracted from sugarcane has had no negative side effects at any time and expresses that positive effects were noted at all stages of digestion.

### EXAMPLE 6

In this example; an individual presenting with multiple digestive disorders including irregularity of bowel movements, periods of gastric reflux, and a lack of satisfaction related to digestion and general wellbeing. Further health issues identified as part of the individuals general health status includes diagnosis at age 67 with Type II Diabetes and a treatment regime involving oral pharmaceuticals (METFORMIN™) to influence Blood Glucose Level (BGL).

While diagnosis with diabetes cannot be directly linked with the cause of digestive issues, the commencement of intestinal problems correlates with the diagnosis of diabetes. Furthermore side effects from the oral pharmaceuticals and general vitamin deficiencies known to occur in diabetics are reported as causing intestinal health issues by the subject.

This individual began trialling the sugarcane fibre described 12 months after initial diagnosis with Type II diabetes. The subject reports that their general health status has benefited greatly from the supplement, increasing overall feelings of well-being. Additionally, a marked improvement to the regularity of bowel movements was reported. Issues related to gastric reflux resulted in a significant reduction in the occurrence and severity of symptoms, described directly by the individual as near elimination of all symptoms.

### EXAMPLE 7

In this example, a 50 year old male with high cholesterol and prior serious cardiovascular events (2 heart attacks) was directed by their medical practitioner to improve their lifestyle, activity and Body Mass Index thus lowering common heart disease risk factors.

The individual made moderately unsuccessful attempts to modify his diet and follow the doctor's advice due to the difficulty of such global changes to lifestyle.

The individual began trialling the sugarcane fibre in accordance with the invention. The dietary fibre was taken as instructed and resulted in a response described as a major improvement in overall wellbeing. Bowel movements became regular with a more satisfying sensation of emptying. Overall dietary consumption was stated as “much less” and a change to the individual's appetite was reported as resulting in cravings toward healthier choices based on the positive outcome in well-being. Weight loss was noted as an outcome and improvement to cholesterol followed the path of correction, targeting the risk factors that the individual was not able to effect prior to the usage of the dietary extract from sugarcane.

This individual considers the dietary fibre extracted from sugarcane a core part of their dietary needs with practitioners satisfied that the changes to his blood test results and psychological approach to his historical health difficulties are linked to the dietary fibre material.

### EXAMPLE 8

In this example a 60+ year old individual suffered a health event triggered by dental work that went undiagnosed and untreated for several months. The result was weight loss and serious concerns for general health. Health remained poor and no cause was identified for over two years.

Symptoms included weight loss, digestive issues and constant exhaustion. Eventual diagnosis revealed a bacterial infection that had affected the heart resulting in systemic health issues in all major areas including digestive health. After treatment to resolve the infection the individual began using the dietary fibre extracted material according to the invention as part of a recovery strategy.

The subject reported an improved feeling of well-being and amelioration of all digestive and diet related issues. The supervising physician agrees that recovery was successful and the subject has returned to standard health check-ups rather than intense treatment.

It will be appreciated by persons skilled in the art that the above described embodiments are not the only ways in which the invention can be put into practice. There are other alternative embodiments which, while different in some details, nevertheless fall within the scope of the invention.

1. Use of dietary fibre material extracted from sugarcane in the manufacture of a food product that is formulated to ameliorate the effects of intestinal tract disorders such as Coeliac's disease and Irritable Bowel Syndrome (IBS).

2. The method of claim 1, wherein the sugar cane fibre is prepared via a process including the steps of: subjecting the sugar cane material to at least one wet diffusion step to separate sugars from a residual fibre material; and subjecting the residual fibre material to a rapid, low-heat drying process thereby to enhance the water retention properties of said residual fibre product.

3. The method of claim 2, wherein the pH of the extraction liquid is held between 6.5 and 7.5.

4. The method of claim 2, wherein the wet extraction step is a counter-current extraction.

5. The method of claim 4, wherein the counter-current extraction step is done under relatively low-shear conditions.

6. The method of claim 2, wherein the wet extraction step is carried out at a temperature in the range 25° C. to 70° C.

7. The method of claim 2, wherein the sugar cane fibre material has undergone a pressure heating step at a pressure in the range 100 psi to 140 psi.

8. The method of claim 7, wherein the temperature of the pressure heating step is in the range 100° C. to 250° C.

9. A food product formulated to ameliorate the effects of intestinal tract disorders such as Coeliac's disease and IBS; said food product containing dietary fibre material extracted from sugarcane.

10. The food product of claim 9, wherein the sugar cane fibre is prepared via a process according to claim 2.

11. The food product of claim 9, wherein said food product is in the form of a drink.

12. The food product of claim 9, wherein, said food product is in the form of a dry powder that can be added to other food products.

13. The food product of claim 9, wherein, said food product is in the form of a pudding.

14. The food product of claim 9, wherein, said food product is in the form of a semi-solid dairy food such as yoghurt.

15. Treatment of the effects of intestinal tract disorders such as Coeliac's disease and IBS in a person by feeding to said person a food product incorporating dietary fibre material extracted from sugarcane.

16. The method of treatment of claim 15, wherein the food product is as defined in claim 9.
